(19) United States
(12) Patent Application Publication
(10) Pub. No.: US 2010/0222403 A1
(43) Pub. Date: Sep. 2, 2010

(54) Micogel Topical Formulations

(75) Inventors: Marcel Borgers, Knokke (BE); Valerie Vroome, Antwerpen (BE); Geert Cauwenbergh, Plainsboro, NJ (US)

Correspondence Address: Glaxo Smith Kline;c/o The Nath Law Group, 112 South West St., Alexandria, VA, 22314-2825, US

(73) Assignee: Barrier Therapeutics, Inc

(21) Appl. No.: 11/993,381

(22) PCT Filed: Jun. 27, 2006
(86) PCT No.: PCT/US06/25194

§ 371 Date: Feb. 4, 2010

Related U.S. Application Data
(60) Provisional application No. 60/693,924, filed on Jun. 27, 2005.

(51) Int. Cl. A61K 31/4174; A61P 31/10; A61P 31/04; A61P 17/10

(52) U.S. Cl. 514/399

(57) ABSTRACT

The present invention encompasses anhydrous micogel formulations containing miconazole and methods of use for the treatment of topical disorders including acne.

### FIELD OF THE INVENTION

The present invention relates to pharmaceutical compositions suitable for topical administration for the treatment of acne and related disorders.

### BACKGROUND OF THE INVENTION

Acne is a common inflammatory pilosebaceous disease characterized by comedones, papules, pustules, inflamed nodules, superficial pus-filled cysts, and (in extreme cases) canalizing and deep, inflamed, sometimes purulent sacs. Acne involves an interaction between hormones, keratinization, sebum, and bacteria that somehow determines the course and severity of acne. It often begins at puberty, when the increase in androgens causes an increase in the size and activity of the pilosebaceous glands. The earliest microscopic change is thought to be intrafollicular hyperkeratosis, which leads to blockage of the pilosebaceous follicle with consequent formation of the comedo, composed of sebum, keratin, and microorganisms, particularly Propionibacterium acnes. Lipases from P. acnes break down triglycerides in the sebum to form free fatty acids (FFA), which irritate the follicular wall. Retention of sebaceous secretions and dilation of the follicle may lead to cyst formation. Rupture of the follicle, with release of FFA, bacterial products and keratin constituents into the tissues, includes an inflammatory reaction that may result in abscess that heals with scars in severe cases. When the condition is less severe, it may merely involve an occasional isolated pimple. However, the underlying pathology is similar to that described above.

Acneiform eruptions can occur wherever there is a pilosebaceous unit or sebaceous follicle which does include the entire surface of the skin. The basic lesion in acne is the comedo commonly known as the blackhead. The comedo is created by retention of layers of dead skin known as keratin in the lining of the follicles. In addition to hyperkeratosis (which is thickening or retentative layering of keratin), there is an accumulation of sebum which is the lipid-laden product of the sebaceous gland. The cells of the sebaceous glands in which sebum originates are the sebocytes. The combination of the keratin and the sebum produces a plugging of the mouth or opening of the follicular canal, and papules are formed by inflammation around the comedones (plural of comedo). Depending upon the degree of inflammation, pustules, cysts, nodules, granulomatous reactions, scars, and keloids may develop.

Most typical forms of mild acne vulgaris demonstrate the predominance of comedones with the occasional pustules. Pustules and papules predominate in more severe cases. These can heal with scar formation; that is, fibrosis of the lesions which are deep and penetrating. In moderately active cases, larger cystic lesions can develop. Acne vulgaris can appear in many clinical varieties. The mildest case manifests comedones on oily skin and is called acne comedo.

Papular acne is another variety of acne which has many inflammatory papules. This form of acne is common in adolescent skin, but it can be seen in all ages. The papular inflammatory form of acne can progress to an indurated, deeper, and destructive form known as acne indurata. These lesions can produce severe scarring and can be quite deep seated and destructive.

Steroid acne vulgaris can occur when oral corticosteroids or topical steroids are used and occurs as inflammatory follicular papules. When oral corticosteroids are ingested, the inflammatory papules are usually sudden in appearance and can cover the chest, back, arm, and face. When topical corticosteroids are used for more than two weeks, a localized inflammatory apular response can develop which can proceed to a granulomatous chronic reaction known as steroid acne rosacea.

Premenstrual acne can occur in a large number of menstruating women as a papular and pustular acne vulgaris, approximately one week prior to menstruation. There is a body of evidence that implicates a surge in progesterone as the mediator of premenstrual acne.

Preadolescent acne is divided into neonatal, infantile, and childhood forms of acne. The neonatal form is limited to the first few weeks of life. It usually develops a couple of days after birth. It more commonly afflicts males and reveals transient facial papules and pustules which can clear spontaneously in a few days or weeks. The stimulation of neonatal sebaceous glands by circulating maternal progesterone appears to be the cause.

If the acne persists beyond the first month of life, the acne is called infantile acne and can extend into childhood, adolescence, and adult life. The childhood acne can result from a persistent infantile acne or can develop de novo after age two. This form of acne is uncommon, but it has more of a male predilection. It is characterized by comedones commonly in groups, papules, pustules, and, rarely, cysts. This condition can extend from a few weeks to several years and can develop into pubertal acne.

Acne venenata is by definition a comedonal or papular acne which occurs after exposure to chlorinated hydrocarbons (chloracne), cutting oils, petroleum oil, coal tar, and pitches. Acne cosmetica is a persistent low grade comedonal and/or papular and pustular acne that occurs usually on the chin and cheeks of adult women due to oil-based cosmetics, i.e., foundations, facial creams, and sunscreens.

Pomade acne is a type of acne cosmetica which appears to occur when grease and oil are applied to scalp hair and to the face as a grooming aid. The lesions are predominately comedonal acne and can develop into inflammatory acne papules, depending upon the chronicity of the pomade use.

Acne detergicans occurs as a type of comedonal acne in patients who use oil-based cleansing soaps. Acne excoriee, also known as pickers acne, starts out as a mild form of papular or comedonal acne which when manipulated or picked, causes further inflammation, more papules, and sometimes scars, pitting, and atrophy of the skin.

Gram negative acne, sometimes called gram negative folliculitis when it extends to the neck, arms, legs, and trunk, is a form of an inflammatory papular, follicular, and pustular response to gram negative organisms including Enterobacter, Klebsiella, Escherichia, Proteus, Serratia and Pseudomonas. The most characteristic lesion on the face are superficial pustules, or papulo-pustules (which is a combination of a papule and pustule). The face can show diffuse erythema and inflammation surrounding these pustules and juicy papules or papulo-pustules.

The gram negative acne is usually highly resistant and usually occurs in patients who have bad inflammatory papular acne for long periods or who have been treated with long term oral administration of antibiotics such as tetracycline, erythromycin, or minocycline or topical antibiotics such as topical clindamycin or topical erythromycin.

Acne rosacea is an inflammatory eruption that is chronic and occurs on the face, especially on the nose as well as the scalp and neck, in some instances. It is manifested by erythema, pustules, papules, telangiectasia (which is dilation of superficial capillaries), and hypertrophy of sebaceous glands. The middle portions of the face are most frequently involved. The eyes and eyelids are not uncommonly involved and can produce inflammation and infection of the conjunctiva, eyelids, and hypertrophy of the meibomian glands. Acne rosacea is often simply called rosacea and is most common in middle aged women and men. Rosacea can go on to form a granulamatous rosacea which is characterized by resistant inflammatory papules which when biopsied reveal non-caseating epithelial cell granulomas.

Pseudofolliculitis barbae is a predominantly male affliction which is characterized by inflammatory papules and pustules on the bearded area of the face. The mechanism is thought to be an inflammatory response to the end of hair (usually curly beard facial hair) into the skin causing a foreign body inflammatory response.

Folliculitis is an inflammatory reaction around the hair follicle which can be bacterial or non-bacterial in nature. Predominately, folliculitis is caused by gram positive organisms such as Staphylococcus and Streptococcus, and less frequently by gram negative bacteria discussed hereinabove with respect to gram negative folliculitis.

Perioral dermatitis is a common papular inflammatory eruption which is confined around the mouth. It most commonly afflicts women in their early twenties to middle thirties, but it can be seen in adolescents and more mature adults. Hiddradenitis suppurativa is a suppurative (chronic) and cystic disease of apocrine gland regions of the skin, including the axillae, perineum and groin.

The etiology of acne vulgaris and related disorders as discussed above is not completely known in every detail. However, what is known is that acne, in general, is caused by a plurality of factors. In general, there are four main factors that cause acne: genetics; hormonal activity; bacteria; and the inflammatory response.

Genetics is a prominent component as it is well known that several members of the same family can be affected with moderate to severe scarring acne. The inheritance by some is thought to be autosomal dominant, but this has not been definitively proven. Furthermore, on the molecular level, there has not yet been discovered a gene or group of genes that are responsible for the various forms of acne vulgaris.

Another key factor in the development of acne is hormonal. In adolescence, for example, it is thought that androgens can interact with receptors on the sebaceous glands and cause stimulation of the sebaceous gland, to hypertrophy and hence form more sebaceous production of lipids and free fatty acids which distend the follicular canal. More specifically, there is evidence at the level of the skin in acne patients. It is further hypothesized that receptors on the sebaceous gland for the active androgen dihydrotestosterone can exhibit various degrees of sensitivity, and that a heightened sensitivity response may be partially or entirely genetically predetermined.

Another causative factor in acne is the presence of bacteria in the follicular canal. Within the follicular canal are bacteria which are indigenous to the follicular lining. Among the bacteria flora present are anaerobic, gram positive organisms called Propionibacterium acnes. It is interesting to note that they are present in abundance in pathologically affected sites. They are reduced during oral antimicrobial treatment, and their absence from nonhuman animal skin is striking especially since animals do not exhibit acne vulgaris.

Yet another causative factor in acne is the inflammatory response manifested in the skin. More specifically, it is thought that Propionibacterium acnes lives in symbiosis on the keratin lined follicular canal. Propionibacterium acnes ingest the sebum produced from the sebocytes of the sebaceous glands. This nascent sebum is largely lipid in composition and also contains DNA, RNA, proteins, and other cellular components that result from the breakdown of sebocytes themselves. The Propionibacterium acnes which are highly lipophilic, feed on the nascent sebum. It has been shown that Propionibacterium acnes are found only in sebaceous rich areas. If the nutrients increase due to an active and large sebaceous system, then colonization and high growth rates of Propionibacterium acnes will form. It has been shown that the resident bacterial flora will produce biologically active molecules such as histamine, extracellular enzymes, and peptides which may be responsible for the chemotaxis of the inflammatory infiltrate in acne vulgaris. Since the follicular lining in the pilosebaceous unit is intact, it has been theorized that if colonization of Propionibacterium acnes occurs in sufficient numbers, they could produce initiating antigenic molecules that promote the initiation of inflammation. Propionibacterium acnes can produce proteinases, lipase, and hyaluronate lyase all of which may serve as the catalysts or initiators of the inflammatory infiltrate which has been shown to be composed of neutrophils and lymphocytes.

A number of treatments are presently known for treating acne, some more successful than others. Some modes of treatment have been mentioned above. There are two modes of treatment, topical and systemic. Aside from treatments mentioned above, some additional systemic treatments for acne that are presently employed are: oral tetracycline; oral erythromycin; minocycline; doxycycline; oral trimethoprim-sulfamethoxazole and isotretinoin.

Some of the topical treatments that are presently employed are: topical erythromycin, clindamycin, benzoyl peroxide, 2% sulfur, 3% resorcinol, a tetracycline derivative (1% meclocycline sulfosalicylate, 2% salicylic acid and tretinoin.

More specifically with respect to the topical use of certain specific antibiotics, a topical solution, ointment, and gel containing erythromycin is used. Also used is a topical solution, gel, and lotion containing clindamycin, and a cream containing 1% meclocycline sulfosalicylate (a tetracycline derivative). Other topical treatments for acne using antibiotics include erythromycin derivatives in conjunction with benzoyl peroxide.

Still other topical treatments for acne using anti-bacterials include an azole derivative in conjunction with benzoyl peroxide (see U.S. Pat. No. 4,446,145, incorporated herein by reference); and metronidazole in a special gel as described in U.S. Pat. No. 4,837,378 incorporated herein by reference. Benzoyl peroxide has been known for several years to be a particularly interesting keratolytic agent among recognized therapeutic acne treatments. In addition, it has good bacteriostatic properties. Benzoyl peroxide-antibiotic combinations, however, are unstable over longer periods of time.

### SUMMARY OF THE INVENTION

The invention encompasses an anhydrous gel composition for topical delivery comprising an alcohol, a glycol, glycerin and miconazole. In one embodiment, the miconazole is solubilized. In other embodiments the alcohol is selected from the group consisting of methanol, ethanol, propanol, isopropanol, isobutanol and combinations thereof while the glycol is selected from the group consisting of polyethylene glycol, propylene glycol and combinations thereof.

In another embodiment, the miconazole is present in the anhydrous gel in an amount of from about 0.1 to about 5 percent by weight, including an amount of from about 0.01 to about 2 percent by weight. The alcohol is present in the anhydrous gel in an amount of from about 10 to about 40 percent by weight. The glycol is present in the anhydrous gel in an amount of from about 20 to about 60 percent by weight, including an amount from about 30 to about 50 percent by weight. The glycerin is present in the anhydrous gel in an amount of from about 10 to about 30 percent by weight, including about 15 to about 25 percent by weight. In yet another embodiment, the anhydrous gel further comprises at least one of an emollient, a viscosifier, a pH adjuster, an antioxidant and/or a colorant.

The invention also encompasses a method of delivering the anhydrous gel composition for the treatment of skin disorders associated with gram positive bacteria or yeasts to a recipient in need thereof comprising topically administering an effective amount of the composition to the recipient in need thereof. In one embodiment, the recipient is a human suffering from skin disorders associated with the following microorganisms: P. acnes, S. aureus, S. epidermis or Malassezia spp. In some embodiments of the invention, the skin disorders are selected from the group consisting of acne, pityriasis versicolor and seborrheic dermatitis.

According to the methods of the invention, the anhydrous gel composition enhances accumulation of hydrogen peroxide in the skin in amounts effective to treat skin disorders associated with gram positive bacteria or yeasts. Furthermore, the administration of the composition limits the microorganism's ability to neutralize the hydrogen peroxide by inhibiting peroxidase and catalase enzyme activities in the microorganism.

The invention further encompasses a method of altering the lipid production and/or composition of keratinocytes present in a sebaceous gland to an extent effective to treat or prevent acne, pityriasis versicolor, or seborrheic dermatitis comprising topically administering an effective amount of the anhydrous gel composition to a recipient in need thereof, preferably a human. In some embodiments, the lipid composition is altered by increasing the amount of linoleic acid present in the keratinocytes.

The invention also encompasses a method of reducing skin inflammation comprising topically administering an effective amount of the anhydrous gel composition to a recipient in need thereof, preferably a human.

### DETAILED DESCRIPTION OF THE INVENTION

The present invention encompasses pharmaceutical compositions for the topical treatment of acne vulgaris. These compositions are non-irritating and have, compared with compositions known in the art, improved anti-acne activity. The compositions of the invention are anhydrous and comprise miconazole, an alcohol, one or more glycols, and glycerin.

High concentrations of miconazole are typically necessary to produce microbiocidal effects against gram positive bacteria such as P. acnes or S. aureus in patients suffering from persistent skin infections by these species. Currently, such concentrations are not achieved at the site of action due to limitations on the solubility of miconazole in topical formulations such as ointments and lotions. To achieve high concentrations, it is necessary that the miconazole be fully solubilized in order to reach adequate drug levels, not only in the superficial layers of the epidermis but also in the pilosebaceous root canals.

The pharmaceutical compositions of the present invention provide for both the production of reactive oxygen species (e.g., H₂O₂) in microbial organisms with simultaneous inhibition of inherent defense systems in these microorganisms (e.g., peroxidase and catalase enzyme activity) which only occur at relatively high levels of miconazole. Following administration of the pharmaceutical compositions of the present invention, sufficiently high concentrations of miconazole are achieved to produce bacteriocidal concentrations of hydrogen peroxide (H₂O₂) endogenously such that the addition of benzoyl peroxide, currently the most common agent administered with compositions of miconazole, is no longer necessary.

Another unexpected advantage of the pharmaceutical compositions of the invention is the alteration of the lipid concentration of the recipient's keratinocytes and sebocytes, due to the high achieved concentrations of miconazole. As an example, the amount of linoleic acid is increased after miconazole treatment. Since this particular fatty acid is found to be at low levels in sebocytes isolated from patients with acne, and since this condition is known to be responsible for the hyperkeratiosis and clotting of the pilosebaceous gland, treatment of a recipient with a pharmaceutical composition of the invention (“micogel treatment”) leads to normalization of the levels of linoleic acid and thereby, the keratinocyte process.

Thus, the present invention encompasses an anhydrous gel formulation comprising miconazole which is suitable for topical administration and methods of using such formulations for the treatment or prevention of acne and related diseases.

As used herein, “topical administration” refers to the conventional delivery of a topical drug or pharmacologically active agent directly to the skin or mucosa of an individual.

As used herein, the term “miconazole” also includes pharmaceutically acceptable salts, solvates, hydrates, clathrates, polymorphs and prodrugs thereof. In addition, the oligonucleotides of the invention may contain one or more chiral centers and/or double bonds and therefore, exist as stereoisomers, such as double-bond isomers (i.e., geometric isomers), enantiomers, or diastereomers. According to the invention, the chemical structures depicted herein, and therefore the polypeptides of the invention, encompass all of the corresponding enantiomers and stereoisomers, that is, both the stereomerically pure form (e.g., geometrically pure, enantiomerically pure, or diastereomerically pure) and enantiomeric and stereoisomeric mixtures. Enantiomeric and stereoisomeric mixtures can be resolved into their component enantiomers or stereoisomers by methods well known to a person of ordinary skill in this art.

As used herein and unless otherwise indicated, the term “pharmaceutically acceptable” means approved by a regulatory agency of the Federal or a state government or listed in the U.S. Pharmacopeia or other generally recognized pharmacopeia for use in animals, and more particularly in humans.

The term “vehicle” refers to a diluent, adjuvant, excipient, or carrier with which a compound of the invention is administered. Such pharmaceutical vehicles can be, for example, liquids, such as water and oils, including those of petroleum, animal, vegetable or synthetic origin, such as peanut oil, soybean oil, mineral oil, sesame oil and the like. The pharmaceutical vehicles can be saline, methyl cellulose, gum acacia, gelatin, starch paste, talc, keratin, colloidal silica, urea, and the like. In addition, auxiliary, stabilizing, thickening, lubricating and coloring agents may be used. When administered to a patient, the compositions of the invention and pharmaceutically acceptable vehicles are preferably sterile. Suitable pharmaceutical vehicles also include excipients such as starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The present compositions, if desired, can also contain minor amounts of pH buffering agents.

As used herein and unless otherwise indicated, the phrase “pharmaceutically acceptable salt” includes, but is not limited to, salts of acidic or basic groups that may be present in the compositions. Miconazole is capable of forming a wide variety of salts with various inorganic and organic acids. The acids that may be used to prepare pharmaceutically acceptable acid addition salts of such basic compounds are those that form non-toxic acid addition salts, (i.e., salts containing pharmacologically acceptable anions), including, but not limited to, sulfuric, citric, maleic, acetic, oxalic, hydrochloride, hydrobromide, hydroiodide, nitrate, sulfate, bisulfate, phosphate, acid phosphate, isonicotinate, acetate, lactate, salicylate, citrate, acid citrate, tartrate, oleate, tannate, pantothenate, bitartrate, ascorbate, succinate, maleate, gentisinate, fumarate, gluconate, glucaronate, saccharate, formate, benzoate, glutamate, methanesulfonate, ethanesulfonate, benzenesulfonate, p-toluenesulfonate and pamoate (i.e., 1,1′-methylene-bis-(2-hydroxy-3-naphthoate) salts. Compounds included in compositions used in the methods of the invention that are acidic in nature are capable of forming base salts with various pharmacologically acceptable cations. Examples of such salts include alkali metal or alkaline earth metal salts and, particularly, calcium, magnesium, sodium, lithium, zinc, potassium and iron salts.

As used herein and unless otherwise indicated, the term “therapeutically effective” refers to an amount of miconazole or a pharmaceutically acceptable salt, solvate, hydrate, clathrate, polymorph or prodrug thereof able to cause an amelioration of a disease or disorder, or at least one discernible symptom thereof. “Therapeutically effective” also refers to an amount that results in an amelioration of at least one measurable physical parameter, not necessarily discernible by the patient. In yet another embodiment, the term “therapeutically effective” refers to an amount that inhibits the progression of a disease or disorder, either physically (e.g., stabilization of a discernible symptom), physiologically (e.g., stabilization of a physical parameter), or both. In yet another embodiment, the term “therapeutically effective” refers to an amount that results in a delayed onset of a disease or disorder.

As used herein and unless otherwise indicated, the term “prophylactically effective” refers to an amount of miconazole, or a pharmaceutically acceptable salt, solvate, hydrate, clathrate, polymorph or prodrug thereof causing a reduction of the risk of acquiring a given disease or disorder. In one embodiment, the compositions are administered as a preventative measure to an animal, preferably a human, having a genetic predisposition to a disorder described herein (e.g., acne). In another embodiment of the invention, the compositions are administered as a preventative measure to a recipient having a non-genetic predisposition to a disorder disclosed herein. Accordingly, the compositions of the invention may be used for the prevention of one disease or disorder and the concurrent treatment of another.

Suitable pharmaceutical compositions for miconazole and/or other therapeutic agents used in the invention include, for example, any and all types of anhydrous gel formulations. Application of the compositions of the invention may also be by aerosol, e.g., with a propellent such as nitrogen, carbon dioxide, a freon, or without a propellent such as a pump, drops, lotions, or a semisolid such as a thickened composition which can be applied by a swab. In addition, the miconazole gel compositions may be applied as a transdermal patch.

The anhydrous gel formulations of the invention can include any therapeutically inactive components such as, for example, oils, fats, waxes, surfactants, humectants, thickening agents, antioxidants, viscosity stabilizers, chelating agents, buffers, preservatives, perfumes, dyestuffs, lower alcohols and the like. If desired, additional ingredients may be incorporated in the compositions of the invention such as antiinflamatory agents, antibacterials, antifungals, disinfectants, vitamins, sunscreens, antibiotics or anti-acne agents.

The pharmaceutical compositions of the invention may also optionally include other carriers, stabilizers, preservatives or adjuvants. For typical examples of these classes of compounds, see Remington: The Science and Practice of Pharmacy, Lippincott (2000), which is incorporated by reference in its entirety.

In one embodiment, the anhydrous gel comprises an alcohol and a polyol, including glycols. Suitable alcohols include, but are not limited to, methanol, ethanol, propanol, isopropanol, isobutanol or combinations thereof. Suitable glycols include, but are not limited to, polyethylene glycol, propylene glycol, ethylene glycol, a sugar or combinations thereof. The alcohol may be present in an amount of from about 5 to about 50 weight percent, with preferred amounts of from about 10 to about 40 weight percent and a more preferred amount of about 35 weight percent. Similarly, the glycol may be present in an amount of from about 30 to about 50 weight percent, with preferred amounts of from about 25 to about 55 weight percent and a more preferred amount of about 40 weight percent.

An effective amount of the miconazole in the combination therapy composition or administered alone ranges from about 0.01 weight percent to about 5 weight percent, more preferably from about 0.01 to about 2 weight percent, and even more preferably from about 0.01 to about 1 weight percent or from about 0.1 to about 3 weight percent. It may be appropriate to administer the miconazole compound, either alone or in a combination therapy, once daily or as two, three, four or more sub-doses at appropriate intervals throughout the day. Sub-doses may be formulated as unit dosage forms, for example, containing 0.001 mg to 500 mg of active ingredient per unit dosage form.

The exact dosage and frequency of administration depends on the particular therapeutic agent being used, the particular disease state being treated, the severity of the disease state being treated, the age, weight and general physical condition of the particular recipient as well as other medication the recipient may be taking, as is well known to those skilled in the art. Furthermore, it is evident that the effective frequency of administration of the combination therapy may be lowered or increased depending on the response of the treated recipient and/or depending on the evaluation of the physician prescribing the therapeutic agent being used.

Additional therapeutic agents which can be combined with the miconzole-containing anhydrous gel include, but are not limited to, antimicrobial agents (e.g, amphotericin B, clotrimazole, econazole nitrate, fluconazole, flucytosine, haloprogin, itraconazole, ketoconazole and nystatin), anti-allergic agents (e.g., astemizole, betamethasone, carbinoxamine maleate, chlorpheniramine maleate, clemastine fumarate, dexbrompheniramine maleate, dexchlorpheniramine maleate, diphenhydramine hydrochloride, diphenylpyraline hydrochloride and trimeprazine tartrate), anti-inflammatory agents (e.g., ibuprofen, fenoprofen, ketoprofen, naproxen, diclofenac, etodolac, meclofenamate sodium phenylbutazone, indomethacin, piroxicam, sulindac and tolmetin), anti-proliferating agents (e.g., mycophenolate mofetil and evodiamine), anti-acne agents (e.g., tretinoin, isotretinoin, salicylic acid, benzoyl peroxide and azaleic acid), anti-pruritic agents (e.g., azelastine, cetirizine permethrin and lindane), anti-aging agents and combinations thereof.

### Methods of Prevention and Treatment of Acne and Related Disorders

Any affected skin area, such as the face, neck, back, or chest of a human, may be topically treated with the anhydrous gel formulation of the present invention. The method may be employed for the prevention and/or treatment of acne and is useful for any variation of acne, whether mild or severe or somewhere in between. The anhydrous gel formulation may be employed for the prevention and/or treatment of disorders of the skin related to acne (“acne related disorder”) including, but not limited to, rosacea, pityriasis versicolor and seborrheic dermatitis. Furthermore, the anhydrous gel formulation works similarly well in the treatment of both males and females who have acne or a related disorder.

As used herein, the term “acne” includes all types of acne in all stages, including acne vulgaris observed in adolescents, acne observed in endocrinologic conditions characterized by excess androgen secretion, and the like, in the active inflammatory (pustule-forming, papule-forming, comedone-forming) and non-inflammatory (blackhead-forming and cyst-forming) phases, and post-inflammatory (healing, scarring and scarred) phase. Examples of acne include, but are not limited to, papular acne, steroid acne vulgaris, premenstrual acne, preadolescent acne, acne venenata, acne cosmetica, pomade acne, acne detergicans, gram negative acne and acne rosacea.

As used herein, the term “acne related disorders” includes, but is not limited to, rosacea, pityriasis versicolor, seborrheic dermatitis, pseudofolliculitis barbae, folliculitis, perioral dermatitis and hiddradenitis suppurativa.

For the treatment of androgenic acne, the compositions are suitable for topical administration. Topical pharmaceutical compositions are preferably in the form of a anhydrous gel formulation adapted for application to the skin. Topical pharmaceutical compositions useful in the method of treatment of the present invention may include about 0.001% to 0.1% of the active compound in an anhydrous gel formulation comprising any pharmaceutically acceptable carrier.

Pharmaceutical compositions of the present invention may be administered in a single daily dose, or the total daily dosage may be administered in divided doses of two, three or four times daily. The pharmaceutical compositions for the present invention can be administered directly to the skin, or via transdermal routes, using those forms of transdermal skin patches well known to those of ordinary skill in that art. To be administered in the form of a transdermal delivery system, the dosage administration will, of course, be continuous rather than intermittent throughout the dosage regimen.

The dosage regimen utilizing the compounds of the present invention is selected in accordance with a variety of factors including type, species, age, weight, sex and medical condition of the recipient along with the severity of the condition to be treated. A physician of ordinary skill can readily determine and prescribe the effective amount of the drug required to prevent, counter, arrest or reverse the progress of the condition. Optimal precision in achieving concentration of drug within the range that yields efficacy without toxicity requires a regimen based on the kinetics of the drug's availability to target sites. This involves a consideration of the distribution, equilibrium, and elimination of a drug.

Without further description, it is believed that one of ordinary skill in the art can, using the preceding description and the following illustrative examples, make and utilize the compounds of the present invention and practice the claimed methods. The following working examples describe embodiments of the present invention, and are not to be construed as limiting in any way the remainder of the disclosure.

### EXAMPLES

### Example 1

### Micogel Formulation

| Component | Weight % |
|---|---|
| Miconazole nitrate | 2.00 |
| Polyethylene Glycol 400 | 20.00 |
| Propylene Glycol | 20.00 |
| Glycerin | 20.00 |
| PPG-15 Stearyl Ether | 2.00 |
| Hydroxypropyl Cellulose | 1.50 |
| Ascorbic Acid | 0.30 |
| Butylated Hydroxytoluene | 0.10 |
| Citric Acid, Monohydrate | 0.10 |
| Ethanol (200 proof) (q.s.) | 34.00 |
| Total | 100 |

### Example 2

### Treatment of Acne with Micogel Formulation

The effect of the 2% miconazole gel formulation described in Example 1 was evaluated in the treatment of facial acne. This double-blind, single center pilot study was conducted as previously described (Petit et al. (2004) Skin Res. Technol. 10, 278-282). A total of 8 male students aged from 20 to 25 years were enrolled. All the patients complained of mild acne corresponding to grades 2 or 3 according to the Leeds revised acne grading system (O'Brien et al. (1998) J. Dermatol. Treat. 9, 215-220). They entered a 10-week split-face assessment of the effect of 2% miconazole (MCZ) in a gel formulation compared to the unmedicated base. Concomitant treatments targeting acne were prohibited. The test medications were applied twice daily on randomized hemifaces.

Acne papules were counted every second week (W0, W2, W4, W6, W8, W10). Their redness was measured using a Visi-Chroma (Biophotonics). The median value of the parameter a* of all the papules was calculated. Cyanoacrylate skin surface strippings (CSSS) were used to collect follicular casts. They were collected from both cheeks using plastic strips (3S Biokit, C+K electronic, Cologne) coated with cyanoacrylate adhesive (Superglue®, Loctite). Image analysis was performed on these CSSS following the comedometry procedure in order to assess the microcomedo numerical density and their cumulative size/cm2 of skin surface. An indication of the amount of P. acnes was also determined on the same CSSS by detecting the porphyrin-induced follicular fluorescence. The percentage of fluorescence follicular casts seen under fluorescence microscopy was calculated.

The individual data obtained at each point of treatment are seen in the following 5 tables. In the following tables, a comma is equivalent to a decimal point.

Median a* value of acne papules.xls

| | MCZ | BASE | MCZ | BASE | MCZ | BASE | MCZ | BASE | MCZ | BASE | MCZ | BASE |
|---|---|---|---|---|---|---|---|---|---|---|---|---|
| VOLUNTEERS | W0 | | W2 | | W4 | | W6 | | W8 | | W10 | |
| 1 | 14.2 | 15.7 | 14.4 | 17.4 | 13.0 | 16.2 | 11.3 | 15.8 | 12.4 | 17.0 | 13.2 | 15.8 |
| 2 | 18.0 | 16.6 | 16.3 | 16.2 | 15.5 | 13.2 | 14.1 | 18.6 | 13.2 | 18.6 | 11.4 | 17.2 |
| 3 | 20.4 | 17.3 | 17.1 | 16.1 | 16.2 | 15.3 | 17.2 | 20.4 | 15.1 | 20.4 | 12.0 | 18.1 |
| 4 | 23.3 | 21.2 | 21.5 | 18.6 | 18.1 | 19.1 | 13.5 | 19.5 | 14.8 | 19.5 | 11.3 | 22.4 |
| 5 | 16.5 | 18.0 | 14.0 | 20.2 | 12.3 | 16.7 | 12.2 | 18.1 | 11.5 | 18.1 | 10.7 | 14.5 |
| 6 | 15.1 | 17.8 | 16.3 | 19.4 | 13.1 | 17.3 | 14.1 | 14.7 | 10.7 | 14.7 | 11.1 | 13.6 |
| 7 | 19.7 | 19.5 | 15.3 | 18.1 | 10.5 | 18.0 | 11.4 | 16.9 | 13.3 | 16.9 | 13.6 | 17.0 |
| 8 | 21.0 | 20.1 | 21.1 | 24.2 | 18.2 | 23.1 | 15.4 | 19.9 | 13.0 | 19.9 | 11.5 | 14.1 |
| MEAN | 18.53 | 18.28 | 17.00 | 18.78 | 14.61 | 17.36 | 13.65 | 17.99 | 13.00 | 18.14 | 11.85 | 16.59 |
| SD | 3.13 | 1.85 | 2.85 | 2.62 | 2.82 | 2.92 | 2.03 | 2.03 | 1.50 | 1.89 | 1.03 | 2.85 |
| MEDIAN | 18.85 | 17.9 | 16.3 | 18.35 | 14.3 | 17 | 13.8 | 18.35 | 13.1 | 18.35 | 11.45 | 16.4 |

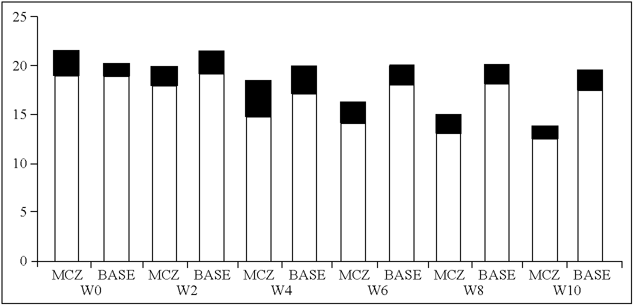

Number of microcom edones/cm2.xls

| | MCZ | BASE | MCZ | BASE | MCZ | BASE | MCZ | BASE | MCZ | BASE | MCZ | BASE |
|---|---|---|---|---|---|---|---|---|---|---|---|---|
| VOLUNTEERS | W0 | | W2 | | W4 | | W6 | | W8 | | W10 | |
| 1 | 17 | 20 | 19 | 20 | 17 | 19 | 15 | 21 | 12 | 22 | 13 | 23 |
| 2 | 33 | 27 | 31 | 28 | 28 | 27 | 27 | 29 | 22 | 27 | 19 | 25 |
| 3 | 22 | 25 | 23 | 21 | 20 | 22 | 19 | 23 | 18 | 24 | 17 | 23 |
| 4 | 27 | 25 | 24 | 25 | 21 | 22 | 16 | 23 | 13 | 21 | 15 | 20 |
| 5 | 19 | 16 | 18 | 18 | 19 | 20 | 18 | 21 | 15 | 20 | 18 | 18 |
| 6 | 25 | 21 | 28 | 24 | 26 | 23 | 21 | 21 | 19 | 24 | 20 | 23 |
| 7 | 31 | 30 | 33 | 35 | 28 | 34 | 25 | 33 | 21 | 31 | 22 | 27 |
| 8 | 36 | 33 | 34 | 35 | 32 | 36 | 30 | 34 | 28 | 33 | 33 | 35 |
| MEAN | 26.25 | 24.63 | 26.25 | 25.75 | 23.88 | 25.38 | 21.38 | 25.63 | 18.50 | 25.25 | 19.63 | 24.25 |
| SD | 6.78 | 5.53 | 6.18 | 6.50 | 5.33 | 6.41 | 5.42 | 5.53 | 5.26 | 4.71 | 6.09 | 5.15 |
| MEDIAN | 26 | 25 | 26 | 24.5 | 23.5 | 22.5 | 20 | 23 | 18.5 | 24 | 18.5 | 23 |

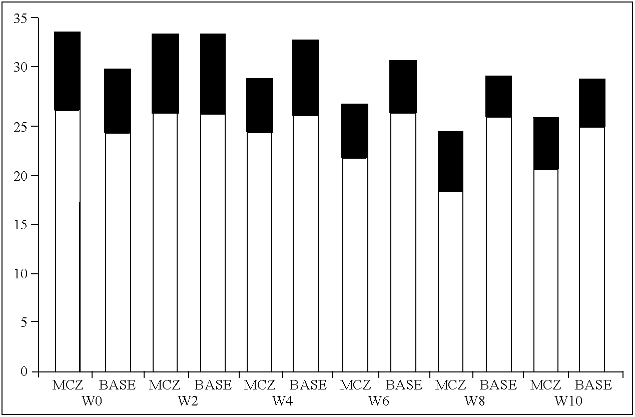

Number of papular acne lesions.xls

| | MCZ | BASE | MCZ | BASE | MCZ | BASE | MCZ | BASE | MCZ | BASE | MCZ | BASE |
|---|---|---|---|---|---|---|---|---|---|---|---|---|
| VOLUNTEERS | W0 | | W2 | | W4 | | W6 | | W8 | | W10 | |
| 1 | 9 | 6 | 9 | 7 | 7 | 8 | 4 | 6 | 6 | 10 | 3 | 8 |
| 2 | 13 | 9 | 11 | 10 | 9 | 10 | 8 | 8 | 6 | 12 | 9 | 10 |
| 3 | 7 | 10 | 6 | 8 | 8 | 7 | 4 | 9 | 2 | 5 | 5 | 11 |
| 4 | 11 | 12 | 10 | 11 | 7 | 14 | 9 | 11 | 9 | 13 | 4 | 12 |
| 5 | 5 | 8 | 9 | 13 | 3 | 11 | 5 | 13 | 3 | 11 | 8 | 8 |
| 6 | 9 | 8 | 11 | 9 | 8 | 10 | 5 | 11 | 9 | 13 | 10 | 12 |
| 7 | 9 | 7 | 9 | 8 | 2 | 7 | 4 | 6 | 8 | 10 | 7 | 9 |
| 8 | 12 | 13 | 13 | 16 | 13 | 19 | 16 | 22 | 14 | 17 | 8 | 14 |
| MEAN | 9.38 | 9.13 | 9.75 | 10.25 | 7.13 | 10.75 | 6.88 | 10.75 | 7.13 | 11.38 | 6.75 | 10.50 |
| SD | 2.62 | 2.42 | 2.05 | 3.01 | 3.44 | 4.06 | 4.16 | 5.18 | 3.80 | 3.42 | 2.49 | 2.14 |
| MEDIAN | 9 | 8.5 | 9.5 | 9.5 | 7.5 | 10 | 5 | 10 | 7 | 11.5 | 7.5 | 10.5 |

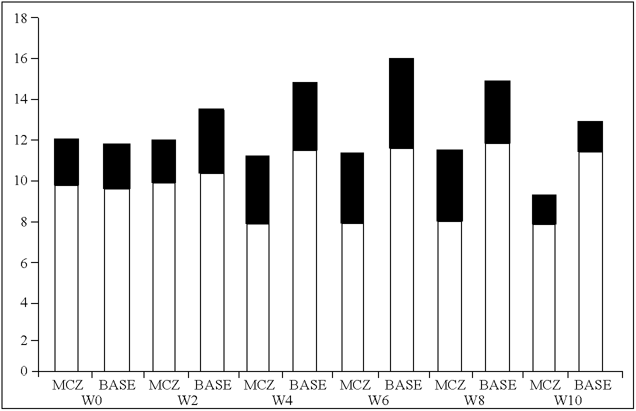

Percentage of fluorescent follicular casts.xls

| | MCZ | BASE | MCZ | BASE | MCZ | BASE | MCZ | BASE | MCZ | BASE | MCZ | BASE |
|---|---|---|---|---|---|---|---|---|---|---|---|---|
| VOLUNTEERS | W0 | | W2 | | W4 | | W6 | | W8 | | W10 | |
| 1 | 15 | 18 | 17 | 21 | 13 | 17 | 8 | 19 | 5 | 17 | 3 | 13 |
| 2 | 7 | 10 | 8 | 13 | 3 | 8 | 2 | 10 | 2 | 8 | 3 | 9 |
| 3 | 6 | 5 | 6 | 3 | 3 | 6 | 5 | 5 | 3 | 7 | 0 | 6 |
| 4 | 9 | 12 | 11 | 10 | 8 | 14 | 2 | 19 | 0 | 23 | 0 | 28 |
| 5 | 13 | 7 | 8 | 8 | 6 | 13 | 5 | 15 | 5 | 12 | 5 | 13 |
| 6 | 8 | 8 | 7 | 10 | 6 | 10 | 6 | 8 | 3 | 8 | 4 | 5 |
| 7 | 14 | 11 | 13 | 12 | 15 | 14 | 12 | 12 | 15 | 14 | 11 | 13 |
| 8 | 22 | 16 | 20 | 23 | 20 | 21 | 17 | 19 | 17 | 20 | 13 | 17 |
| MEAN | 11.75 | 10.88 | 11.25 | 12.50 | 9.25 | 12.88 | 7.13 | 13.38 | 6.25 | 13.63 | 4.88 | 13.00 |
| SD | 5.34 | 4.42 | 5.06 | 6.61 | 6.14 | 4.85 | 5.14 | 5.48 | 6.25 | 5.97 | 4.76 | 7.27 |
| MEDIAN | 11 | 10.5 | 9.5 | 11 | 7 | 13.5 | 5.5 | 13.5 | 4 | 13 | 3.5 | 13 |

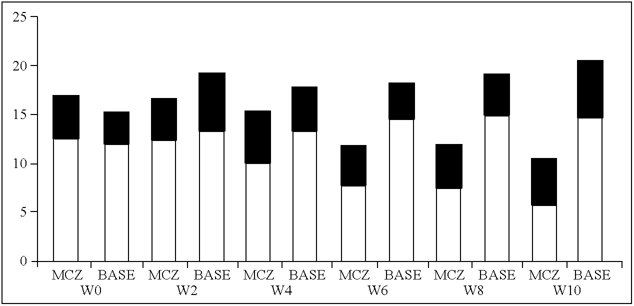

Size of microcom edones/cm 2.xls

| | MCZ | BASE | MCZ | BASE | MCZ | BASE | MCZ | BASE | MCZ | BASE | MCZ | BASE |
|---|---|---|---|---|---|---|---|---|---|---|---|---|
| VOLUNTEERS | W0 | | W2 | | W4 | | W6 | | W8 | | W10 | |
| 1 | 1.7 | 1.4 | 1.8 | 1.5 | 1.7 | 1.8 | 1.4 | 1.6 | 1.1 | 1.5 | 1.4 | 1.7 |
| 2 | 2.9 | 2.6 | 2.6 | 2.7 | 2.7 | 2.5 | 2.3 | 2.5 | 2.0 | 2.3 | 2.1 | 2.2 |
| 3 | 1.8 | 2.0 | 1.6 | 1.9 | 2.0 | 1.8 | 1.7 | 2.1 | 1.6 | 2.1 | 1.8 | 2.3 |
| 4 | 2.2 | 2.4 | 2.1 | 2.6 | 1.8 | 2.4 | 1.3 | 2.3 | 1.1 | 2.2 | 2.0 | 2.5 |
| 5 | 2.3 | 2.1 | 2.4 | 2.3 | 2.1 | 2.3 | 1.6 | 2.2 | 1.3 | 2.0 | 1.5 | 1.9 |
| 6 | 0.9 | 1.2 | 1.1 | 1.1 | 0.7 | 1.2 | 0.9 | 1.4 | 1.1 | 1.6 | 1.3 | 1.6 |
| 7 | 1.5 | 1.3 | 1.3 | 1.4 | 1.2 | 1.4 | 1.3 | 1.8 | 1.2 | 1.8 | 1.2 | 1.6 |
| 8 | 2.0 | 1.7 | 1.9 | 2.1 | 2.0 | 1.8 | 1.6 | 1.9 | 1.4 | 2.0 | 1.5 | 1.9 |
| MEAN | 1.91 | 1.84 | 1.85 | 1.95 | 1.78 | 1.90 | 1.51 | 1.98 | 1.35 | 1.94 | 1.60 | 1.96 |
| SD | 0.59 | 0.52 | 0.52 | 0.58 | 0.60 | 0.47 | 0.41 | 0.37 | 0.32 | 0.28 | 0.33 | 0.34 |
| MEDIAN | 1.9 | 1.85 | 1.85 | 2 | 1.9 | 1.8 | 1.5 | 2 | 1.25 | 2 | 1.5 | 1.9 |

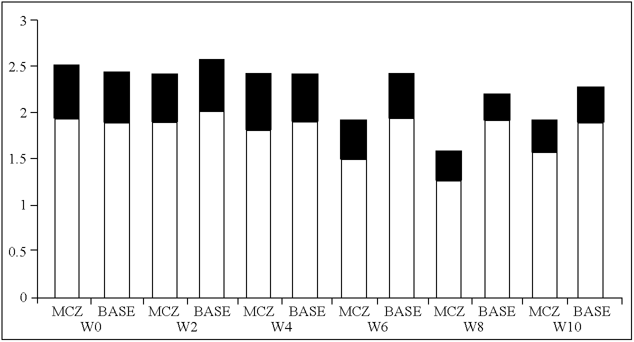

Statistically significant differences between placebo (base) and active (MCZ, micogel) treatments are seen for the following acne lesions at the following time points:

| | | |
|---|---|---|
| Acne papules | Week 4 | p < 0.05 |
| | Week 6 | p < 0.01 |
| | Week 8 | p < 0.01 |
| | Week 10 | p < 0.01 |
| No. Papular lesions | Week 4 | p < 0.05 |
| | Week 6 | p < 0.01 |
| | Week 8 | p < 0.01 |
| | Week 10 | p < 0.05 |
| No. Microcomedones | Week 6 | P < 0.05 |
| | Week 8 | p < 0.001 |
| | Week 10 | p < 0.05 |
| Size Microcomedones | Week 6 | p < 0.01 |
| | Week 8 | p < 0.01 |
| | Week 10 | P < 0.01 |
| Fluorescent follicular casts | Week 4 | P < 0.05 |
| (=presence of P. acnes) | Week 6 | P < 0.05 |
| | Week 8 | p < 0.05 |
| | Week 10 | p < 0.01 |

Reports of irritation following patient treatment with the micogel formulation were minimal to non-existent. Topical application of the micogel is an effective and well tolerated treatment for patients with acne (grades 2 and 3 of the Leeds revised acne grading system).

Unless defined otherwise, all technical and scientific terms herein have the same meaning as commonly understood by one of ordinary skill in the art to which this invention belongs. Although any methods and materials, similar or equivalent to those described herein, can be used in the practice or testing of the present invention, the preferred methods and materials are described herein. All publications and patent applications cited herein are incorporated herein by reference for the purpose of disclosing and describing specific aspects of the invention for which the publication is cited.

1. An anhydrous composition for topical delivery comprising:
(a) an alcohol,
(b) a glycol,
(c) glycerin, and
(d) miconazole
wherein the composition is formulated as an anhydrous gel.

2. The composition according to claim 1 wherein the miconazole is solubilized.

3. The composition according to claim 1 wherein the alcohol is selected from the group consisting of ethanol, isopropanol, isobutanol and combinations thereof.

4. The composition according to claim 1 wherein the glycol is selected from the group consisting of polyethylene glycol, propylene glycol and combinations thereof.

5. The composition according to claim 1 wherein the miconazole is present in an amount of from about 0.01 to about 5 percent by weight.

6. The composition according to claim 1 wherein the miconazole is present in an amount of from about 0.1 to about 2 percent by weight.

7. The composition according to claim 1 wherein the alcohol is ethanol.

8. The composition according to claim 1 wherein the alcohol is present in an amount of from about 10 to about 40 percent by weight.

9. The composition according to claim 1 wherein the alcohol is present in an amount of from about 30 to about 35 percent by weight.

10. The composition according to claim 1 wherein the glycol is present in an amount of from about 20 to about 60 percent by weight.

11. The composition according to claim 1 wherein the glycol is present in an amount of from about 30 to about 50 percent by weight.

12. The composition according to claim 1 wherein the amount of the glycerin is about 10 to about 30 percent by weight.

13. The composition according to claim 1 wherein the amount of the glycerin is about 15 to about 25 percent by weight.

14. The composition according to claim 1 further comprising at least one of an emollient, a viscosifier, a pH adjuster, an antioxidant or a colorant.

15. A method of delivering a composition according to claim 1 for the treatment of skin disorders associated with gram positive bacteria or yeasts to a recipient in need thereof comprising topically administering an effective amount of the composition to the recipient.

16. The method according to claim 15 wherein the recipient is a human.

17. The method according to claim 16 wherein the human is suffering from skin disorders associated with P. acnes, S. aureus, S. epidermis or Malassezia spp.

18. A method of treating skin disorders associated with gram positive bacteria or yeasts comprising topically administering the composition according to claim 1 to a recipient in need thereof.

19. The method according to claim 18 wherein the recipient is a human.

20. The method according to claim 19 wherein the human is suffering from skin disorders associated with P. acnes, S. aureus, S. epidermis, or Malassezia spp.

21. The method according to claim 20 wherein the skin disorders are selected from the group consisting of acne, pityriasis versicolor, and seborrheic dermatitis.

22. A method of treating acne, pityriasis versicolor, and seborrheic dermatitis comprising topically administering an effective amount of the composition according to claim 1 to a recipient in need thereof.

23. The method according to claim 22 wherein the composition enhances accumulation of hydrogen peroxide in microbial organisms in the skin in amounts effective to treat skin disorders associated with grain positive bacteria or yeasts.

24. The method according to claim 23 wherein the administration of the composition limits the microorganism's ability to neutralize the hydrogen peroxide by inhibiting peroxidase and catalase enzyme activities in the microorganism.

25. A method of altering the lipid production and/or composition of keratinocytes and/or sebocytes present in the epidermis or a sebaceous gland to an extent effective to treat or prevent acne, pityriasis versicolor, or seborrheic dermatitis comprising topically administering an effective amount of the composition according to claim 1 to a recipient in need thereof.

26. The method according to claim 25 wherein the lipid composition is altered by increasing the amount of linoleic acid present in the keratinocytes and/or sebocytes.

27. A method of reducing skin inflammation comprising topically administering an effective amount of the composition according to claim 1 to a recipient in need thereof.
